(19) United States
(12) Patent Application Publication
(10) Pub. No.: US 2006/0074042 A1
(43) Pub. Date: Apr. 6, 2006

(54) Highly hypoxia-specific gene expression system for ischemic gene therapy

(75) Inventors: Sung Wan Kim, Salt Lake City, UT (US); Minhyung Lee, Kyunggi-do (KR); Donghoon Choi, Seoul (KR)

Correspondence Address: ALAN J. HOWARTH, P.O. BOX 1909, SANDY, UT, 84091-1909, US

(73) Assignee: University of Utah Research Foundation

(21) Appl. No.: 11/238,664

(22) Filed: Sep. 28, 2005

Related U.S. Application Data
(60) Provisional application No. 60/614,451, filed on Sep. 28, 2004.

(51) Int. Cl. A61K 48/00

(52) U.S. Cl. 514/44; 435/320.1

(57) ABSTRACT

Compositions and methods for treating ischemic diseases, such as ischemic heart disease, are disclosed. Plasmid pEpo-SV-VEGF-EpoUTR comprises an erythropoietin enhancer and an erythropoietin 3′ untranslated region operably coupled to a vascular endothelial growth factor (VEGF) coding segment. High expression of VEGF under hypoxic conditions is obtained. Similar plasmids, with anticancer agents placed under control of hypoxia-regulated elements, can be used for treating solid tumor cancers.

### CROSS-REFERENCE TO RELATED APPLICATIONS

This application claims the benefit of U.S. Provisional Application No. 60/614,451, filed Sep. 28, 2004, which is hereby incorporated by reference in its entirety.

### STATEMENT REGARDING FEDERALLY SPONSORED RESEARCH OR DEVELOPMENT

This invention was made with government support under grant no. R01-HL07154 from the National Institutes of Heath. The government has certain rights in the invention.

### BACKGROUND OF THE INVENTION

This invention relates to gene therapy. More particularly, this invention relates to compositions and methods for treating ischemic diseases and other diseases characterized by localized hypoxic conditions.

Gene therapy with vascular endothelial growth factor (VEGF) is a new treatment for ischemic diseases, such as ischemic heart disease. Therapeutic angiogenesis is a new treatment in cardiovascular disease and hindlimb ischemia. Therapeutic angiogenic therapy is performed by the delivery of the angiogenic agent. VEGF is currently the most effective therapeutic gene for neo-vascularization. J. M. Isner, Myocardial gene therapy, 415 Nature 234-239 (2002). Therapeutic angiogenesis using VEGF gene therapy has been established after preclinical and clinical studies. It was reported previously that both VEGF and its receptors were upregulated in ischemic tissues. G. Brogi et al., Hypoxia-induced paracrine regulation of vascular endothelial growth factor receptor expression, 97 J. Clin. Invest. 469-476 (1996). Therefore, it was suggested that ischemia is necessary for VEGF to enhance its effects. J. S. Lee & A. M. Feldman, Gene therapy for therapeutic myocardial angiogenesis: a promising synthesis of two emerging technologies, 4 Nature Med. 739-742 (1998). Further research proved, however, that exogenously delivered VEGF could exert a physiological effect in normal, non-ischemic tissue. M. L. Springer et al., VEGF gene delivery to muscle: potential role for vasculogenesis in adults, 2 Mol. Cell 549-558 (1998). In addition, unregulated continuous expression of VEGF is associated with formation of endothelial cell-derived intramural vascular tumors. R. J. Lee et al., VEGF gene delivery to myocardium: deleterious effects of unregulated expression, 102 Circulation 898-901 (2000). This suggested that VEGF expression must be regulated to avoid these deleterious effects. Therefore, the erythropoietin (Epo) enhancer was used to enhance VEGF gene expression locally in ischemic tissues. It was also shown that the combination of the Epo enhancer and the SV40 promoter induced gene expression under hypoxia in human embryonic kidney 293 (HEK293) cells in vitro and in rabbit ischemic myocardium in vivo. M. Lee et al., Hypoxia inducible VEGF gene delivery to ischemic myocardium using water-soluble lipopolymer, 10 Gene Ther. 1535-1542 (2003). In addition, other research proved that the hypoxia-responsive element (HRE) mediated VEGF expression in ischemic myocardium using adeno-associated virus as a delivery agent. H. Su et al., Adeno-associated viral vector-mediated hypoxia response element-regulated gene expression in mouse ischemic heart model, 99 Proc. Nat'l Acad. Sci. USA 9480-9485 (2002). In this trial, the VEGF gene was regulated by HRE and the SV40 promoter. This regulation of the VEGF expression system may be useful for safer VEGF gene therapy, minimizing unwanted side effects.

While prior art compositions and methods of use thereof are known and are generally suitable for their limited purposes, they possess certain inherent deficiencies that detract from their overall utility in treating ischemic diseases. For example, unregulated VEGF-mediated angiogenesis has the potential to promote tumor growth, accelerate diabetic proliferative retinopathy, and promote rupture of atherosclerotic plaque.

In view of the foregoing, it will be appreciated that providing a gene delivery composition for regulated VEGF gene expression and methods for treating ischemic diseases would be significant advancements in the art.

### BRIEF SUMMARY OF THE INVENTION

It is a feature of the present invention to provide compositions and methods of use for regulated expression of VEGF in ischemic tissues as a treatment for ischemic diseases.

These and other advantages can be addressed by providing a plasmid comprising a hypoxia-regulated enhancer element operationally configured adjacent to a promoter operable in mammalian cells, an expression cassette encoding vascular endothelial growth factor, and a 3′ untranslated region from a hypoxia-regulated gene, wherein expression of vascular endothelial growth factor in a suitable cell is higher under hypoxia as compared to normal oxygen tension. Illustrative examples of such a hypoxia-regulated enhancer element, promoter operable in mammalian cells, and 3′ untranslated region from a hypoxia-regulated gene comprise an erythropoietin enhancer, an SV40 promoter, and an erythropoietin 3′ untranslated region, respectively. An illustrative example of such a plasmid is pEpo-SV-VEGF-EpoUTR.

Another illustrative embodiment of the present invention comprises a method for treating an ischemic disease, comprising administering to a patient in need of treatment for such ischemic disease a composition comprising a mixture of pEpo-SV-VEGF-EpoUTR and a pharmaceutically acceptable gene delivery carrier. Ischemic heart disease is an illustrative example of such an ischemic disease.

Still another illustrative embodiment of the present invention comprises a method for treating cancer in a patient having a solid tumor, comprising administering a plasmid comprising a hypoxia-regulated enhancer element operationally configured adjacent to a promoter operable in mammalian cells, an expression cassette encoding an anticancer agent, and a 3′ untranslated region from a hypoxia-regulated gene, wherein expression of the anticancer agent in an ischemic region of the solid tumor is higher than in non-ischemic tissues.

Yet other illustrative embodiments of the invention include reporter plasmids pSV-Luc-EpoUTR (SEQ ID NO:5) and pEpo-SV-Luc-EpoUTR (SEQ ID NO:6).

### BRIEF DESCRIPTION OF THE SEVERAL VIEWS OF THE DRAWINGS

FIGS. 1A-D show schematic representations of plasmids pSV-Luc (SEQ ID NO:1), pEpo-SV-Luc (SEQ ID NO:4), pSV-Luc-EpoUTR (SEQ ID NO:5), and pEpo-SV-Luc-EpoUTR (SEQ ID NO:6), respectively.

FIG. 2 shows luciferase expression in the presence and absence of 100 μM CoCl₂. Complexes of pSV-Luc/PEI, pEpo-SV-Luc/PEI, pSV-Luc-EpoUTR/PEI, and pEpo-SV-Luc-EpoUTR/PEI were transfected into human embryonic kidney 293 cells. The transfected cells were incubated in the presence or absence of 100 mM CoCl₂ for 20 hrs. Transgene expression was evaluated by luciferase assay. The data are expressed as mean values (±standard deviation) of three experiments.

FIG. 3 shows luciferase expression under normoxia and hypoxia. Complexes pSV-Luc/PEI, pEpo-SV-Luc/PEI, pSV-Luc-EpoUTR/PEI, and pEpo-SV-Luc-EpoUTR/PEI were transfected into human embryonic kidney 293 cells. The transfected cells were incubated under normoxia (pO₂, 152 nm/mhg) or hypoxia (pO₂, 22.8 mmHg) for 20 hrs. Transgene expression was evaluated by luciferase assay. The data are expressed as mean values (±standard deviation) of four experiments.

FIG. 4 shows a schematic representation of the structure of pEpo-SV-VEGF-EpoUTR.

### DETAILED DESCRIPTION

Before the present compositions and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to a plasmid comprising “a hypoxia-regulated enhancer element” includes reference to one or more of such enhancer-regulated enhancer elements, reference to “an expression cassette encoding vascular endothelial growth factor” includes reference to one or more of such expression cassettes, and reference to “the erythropoietin 3′ untranslated region” includes reference to one or more of such erythropoietin 3′ untranslated regions.

In describing and claiming the present invention, the following terminology will be used in accordance with the definitions set out below.

As used herein, “comprising,” “including” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.” As used herein, “consisting of” and grammatical equivalents thereof exclude any element, step, or ingredient not specified in the claim. As used herein, “consisting essentially of” and grammatical equivalents thereof limit the scope of a claim to the specified materials or steps and those that do not materially affect the basic and novel characteristic or characteristics of the claimed invention.

As used herein, such a “pharmaceutically acceptable gene delivery carrier” is a gene delivery carrier that is suitable for use with humans and/or animals without undue adverse side effects (such as toxicity, irritation, and allergic response) commensurate with a reasonable benefit/risk ratio. Gene delivery carriers include those currently known in the art, such as polyethylenimine and water soluble lipopolymer (WSLP), as well as those that may be developed in the future.

As used herein, “administering” and similar terms mean delivering the composition to the individual being treated such that the composition is delivered to the parts of the body where the composition can encounter hypoxic conditions and be induced to express elevated amounts of the encoded agent, such as VEGF or an anticancer agent. Injectables for such use can be prepared in conventional forms, either as a liquid solution or suspension or in a solid form suitable for preparation as a solution or suspension in a liquid prior to injection, or as an emulsion. Suitable excipients include, for example, water, saline, dextrose, glycerol, ethanol, and the like; and if desired, minor amounts of auxiliary substances such as wetting or emulsifying agents, buffers, and the like can be added.

As used herein, “anticancer agent” means a peptide anticancer agent capable of being expressed from an expression plasmid. Illustrative anticancer agents include asparaginase, interferon alfa, interferon beta, interferon gamma, interleukin-1 alpha and beta, interleukin-3, interleukin-4, interleukin-6, monocyte/macrophage colony-stimulating factor, granulocyte-macrophage colony-stimulating factor, tumor necrosis factor, and the like.

An illustrative embodiment of the present invention relates to a new plasmid, pSV-Luc-EpoUTR (SEQ ID NO:5), which contains the Epo MRNA stabilizer (Epo 3′-UTR). Epo 3′-UTR is known to stabilize Epo mRNA under hypoxia. E. C. McGary et al., Post-transcriptional regulation of erythropoietin MRNA stability by erythropoietin mRNA-binding protein, 272 J. Biol. Chem. 8628-8634 (1997). This Epo 3′-UTR stabilized the chloramphenicol acetyl transferase (CAT) MRNA, when it was inserted downstream of the CAT cDNA. L. E. Huang et al., Regulation of hypoxia-inducible factor 1alpha is mediated by an O2- dependent degradation domain via the ubiquitin-proteasome pathway, 95 Proc. Nat'l Acad. Sci. USA 7987-7992 (1998). In the results described herein, pSV-Luc-EpoUTR was evaluated as a hypoxia inducible gene expression system. In addition, the combination of the Epo enhancer and the Epo 3′-UTR by construction of pEpo-SV-Luc-EpoUTR (SEQ ID NO:6) showed highly specific gene expression under hypoxic conditions. This highly specific gene regulation system for hypoxic conditions will be valuable for gene therapy of ischemic diseases without side effects.

### EXAMPLE 1

Construction of pEpo-SV-Luc

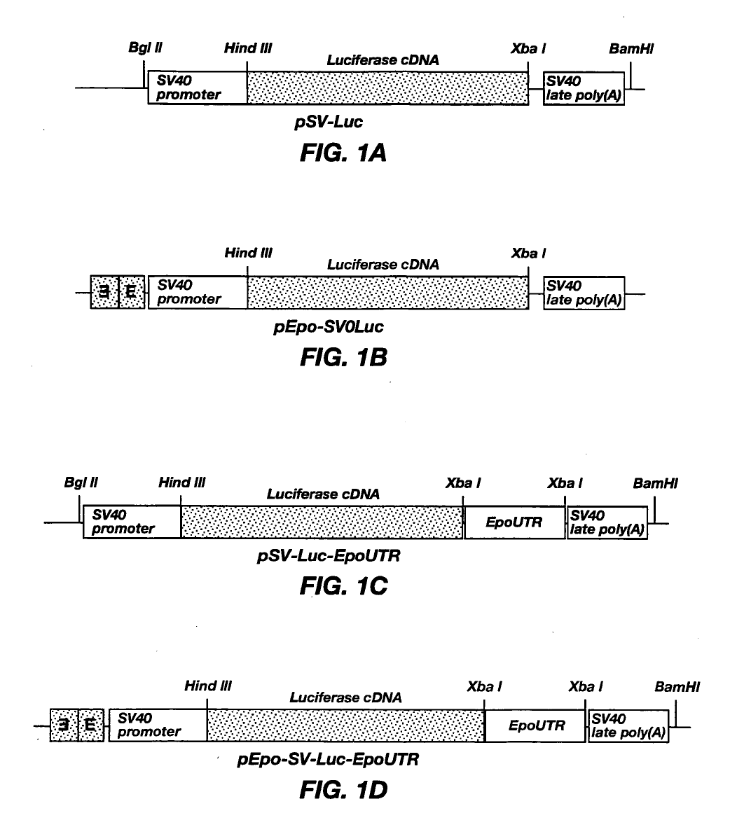

Plasmid pEpo-SV-Luc (SEQ ID NO:4) was constructed as described previously. M. Lee et al., supra. The plasmid referred to herein as pSV-Luc was purchased from Promega (Madison, Wis.) (pGL3-Promoter; SEQ ID NO:1). The Epo enhancer was chemically synthesized according to methods well known in the art. The sequence of the Epo enhancer is as follows: 5′-gccctacgtgctgtctcacacagcctgtctgacctctcgacctaccggcg-3′ (SEQ ID NO:2). XbaI and BamHI restriction endonuclease sites were introduced at each end of the Epo enhancer. The synthesized Epo enhancer was annealed and ligated to produce multiple copies of the Epo enhancer. This ligated Epo enhancer was inserted upstream from the SV40 promoter at the BglII site of pSV-Luc (FIG. 1A), resulting in construction of pEpo-SV-Luc (FIG. 1B). Construction of this plasmid was confirmed by restriction enzyme analysis according to methods well known in the art.

### EXAMPLE 2

Construction of SV-Luc-EpoUTR

The Epo 3′-UTR was cloned by RT-PCR using total RNA from HepG2 cells (ATCC, Manassas, Va.). Total RNA was extracted from HepG2 cells using the RNAwiZh RNA isolation reagent (Ambion, Austin, Tx.). The concentration of RNA was measured by absorbance at 260 nm. Two micrograms of total RNA was used as a template for RT-PCR according to methods well known in the art. XbaI restriction endonuclease sites were added to the primers for cloning convenience. The RT-PCR was performed using the Access RT-PCR system (Promega, Madison, Wis.). The cloned fragment was digested with XbaI and purified by agarose gel electrophoresis and elution. The purified fragment was inserted at the XbaI site of pSV-Luc, resulting in construction of pSV-Luc-EpoUTR (FIG. 1C; SEQ ID NO:5). Construction of this plasmid was confirmed by restriction endonuclease analysis according to methods well known in the art.

### EXAMPLE 3

Construction of pEpo-SV-Luc-EpoUTR

The Epo enhancer was synthesized according to the procedure of Example 1. The synthesized Epo enhancer was annealed and ligated to produce multiple copies of the Epo enhancer. This ligated Epo enhancer was inserted at the BglII restriction endonuclease site of pSV-Luc-EpoUTR (SEQ ID NO:5), resulting in construction of pEpo-SV-Luc-EpoUTR (FIG. 1D; SEQ ID NO:6). Construction of this plasmid was confirmed by restriction endonuclease analysis according to methods well known in the art.

### EXAMPLE 4

In Vitro Transfection Assay

To evaluate the level of the luciferase expression, pSV-Luc (SEQ ID NO:1) , pEpo-SV-Luc (SEQ ID NO:4), pSV-Luc-EpoUTR (SEQ ID NO:5), and pEpo-SV-Luc-EpoUTR (SEQ ID NO:6) were transfected into human embryonic kidney (HEK293) cells using polyethylenimine (PEI) as a gene carrier. HEK293 cells were obtained from ATCC (Manassas, Va.). HEK293 cells were maintained in DMEM medium (R. Dulbecco & G. Freeman, 8 Virology 396 (1959); J. D. Smith et al., 12 Virology 185 (1960)) supplemented with 10% fetal bovine serum (FBS) in a 5% CO₂ incubator. For the transfection assays, the cells were seeded at a density of 5.0×10⁵ cells/well in 6-well flat-bottomed microassay plates (Falcon Co., Becton Dickenson, Franklin Lakes, N.J.) 24 hrs before the transfection. Plasmid/PEI complexes were prepared at a 5/1 N/P ratio, as described previously. M. Lee et al., Sp1-dependent regulation of the RTP801 promoter and its application to hypoxia-inducible VEGF plasmid for ischemic disease, 21 Pharm. Res. 736-741 (2004). The cells were washed twice with serum-free medium, and then 2 ml of fresh serum-free medium was added. The pDNA/PEI complex was added to each well. The cells were then incubated for 4 hrs at 37° C. in a 5% CO₂ incubator. After 4 hrs, the transfection mixtures were removed and 2 ml of fresh medium containing FBS was added. HIF-1 was previously found to be induced by CoCl₂ treatment. G. L. Wang & G. L. Semenza, General involvement of hypoxia-inducible factor 1 in transcriptional response to hypoxia, 90 Proc. Nat'l Acad. Sci. USA 4304-4308 (1993). Therefore, HIF-1 DNA binding activity was induced by cobalt as well as by hypoxia. Hence, the cells were incubated in 100 mM CoCl₂ or in 3% oxygen (pO₂, 22.8 mmHg) for an additional 20 hrs for hypoxic conditions. The control cells were incubated without CoCl₂ or in 20% oxygen (pO₂, 152 mmHg). The cells were harvested for luciferase assay.

After incubation, the cells were washed twice with phosphate-buffered saline (PBS), and 200 μl of reporter lysis buffer (Promega Cat. No. E3971, Madison, Wis.) was added to each well. After 15 min of incubation at room temperature, the cells were harvested and transferred to microcentrifuge tubes. After 15 s of vortexing, the cells were centrifuged at 11,000 rpm for 3 min. The extracts were transferred to fresh tubes and stored at −70° C. until use. The protein concentrations of the extracts were determined using a BCA (bicinchoninic acid) protein assay kit (U.S. Pat. No. 4,839,295; Pierce Chemical Co., Iselin, N.J.). Luciferase activity was measured in terms of relative light units (RLU) using a 96-well plate Luminometer (Dynex Technologies Inc, Chantilly, Va.). The luciferase activity was monitored and integrated over a period of 30 sec. The final values of luciferase were reported in terms of RLU/mg total protein.

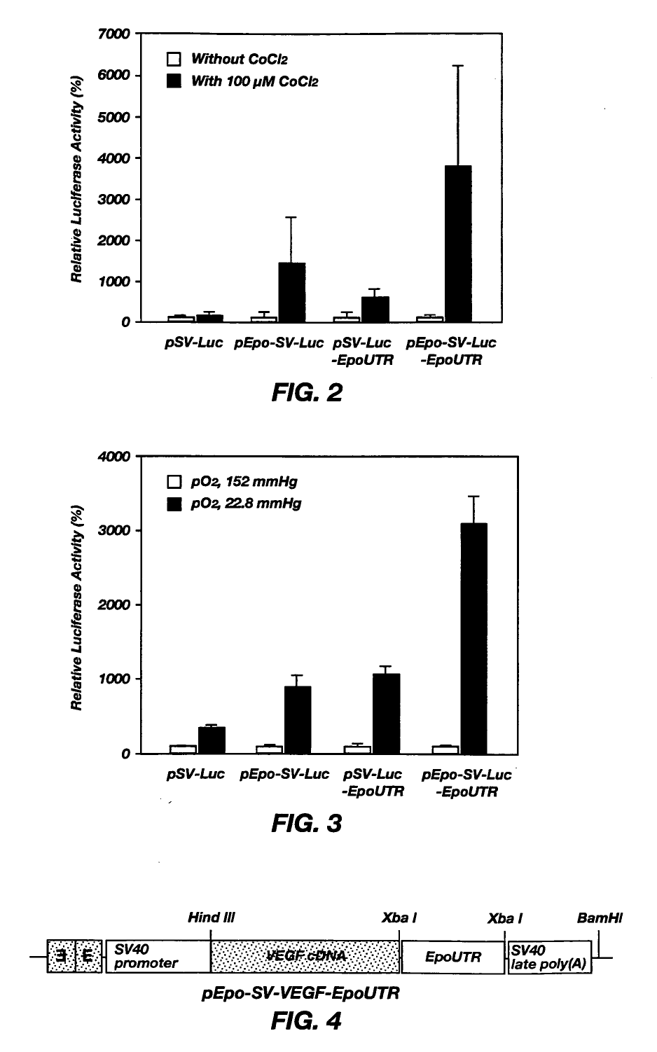

Plasmid pEpo-SV-Luc induced luciferase expression in the presence of cobalt, confirming previous results (FIG. 2). M. Lee et al., 10 Gene Ther. 1535-1542 (2003). Also, pSV-Luc-EpoUTR induced luciferase expression in the presence of cobalt (FIG. 2). The combination of the Epo enhancer and Epo 3′-UTR further enhanced gene expression. As shown in FIG. 2, pEpo-SV-Luc-EpoUTR induced luciferase expression over 30 times in the presence of cobalt. However, pSV-Luc did not show this effect in the presence of cobalt (FIG. 2). This result suggests that the combination of the Epo enhancer and Epo 3′-UTR highly and specifically induces transgene expression under hypoxic conditions.

This result was confirmed by incubation of the cells under hypoxic condition. After transfection, the cells were incubated under normoxia (20% O₂) or hypoxia (3% O₂) for 20 hrs. After 20 hrs of incubation, pEpo-SV-Luc expressed approximately 10 times more luciferase protein under hypoxia than under normoxia (FIG. 3). Also, pSV-Luc-EpoUTR expressed approximately 11 times more luciferase protein under hypoxia than normoxia (FIG. 3). Without the Epo enhancer or the Epo 3′-UTR, pSV-Luc only slightly increased the production of luciferase under hypoxia (FIG. 3). The combination of the Epo enhancer and the Epo 3′-UTR induced luciferase expression over 30 times under hypoxic conditions, compared to normoxic conditions (FIG. 3). Therefore, induction of gene expression by the combination of the Epo enhancer and the Epo 3′-UTR was highly specific for hypoxic cells.

### EXAMPLE 5

Construction of pEpo-SV-VEGF-EpoUTR

Therapeutic angiogenesis with VEGF gene delivery is a potential treatment in cardiovascular disease and hindlimb ischemia. However, unregulated VEGF-mediated angiogenesis has the potential to promote tumor growth, accelerate diabetic proliferative retinopathy, and promote rupture of atherosclerotic plaque. The Epo enhancer/3′-UTR combination system of the present invention is highly specific under hypoxia conditions for gene expression. Therefore, the present invention can be applied to hypoxia-specific VEGF gene therapy while removing or reducing potential risks. The hypoxia-specific VEGF plasmid using the Epo enhancer/3′-UTR combination system is constructed as shown in FIG. 4 by replacing the luciferase coding sequence of pEpo-SV-Luc-EpoUTR (SEQ ID NO:6) with the VEGF coding sequence (SEQ ID NO:3), resulting in pEpo-Luc-EpoUTR (SEQ ID NO:7).

### EXAMPLE 6

Application of the Epo Enhancer/3′-UTR Combination System to Treating Iscbemic Disease

Rabbits are a new model of chronic cardiac ischemia. C. Operschall et al., A new model of chronic cardiac ischemia in rabbits, 88 J. Appl. Physiol. 1438-1445 (2000). Male New Zealand white rabbits weighing 2.5-3.0 kg are medicated with Ketamine (25 mg/kg) and Xylazine (4.4 mg/kg) prior to shaving the left chest. Isoflurane is administered via an endotracheal tube during the operation. The depth of the anesthesia is adjusted by corneal reflex and muscle tone to an adequate level for the surgery. The small animal ventilator is set for a rate of 40 breaths/min and a volume of 30 ml, which is adjusted once the lungs are seen to fill the lungs with each breath.

The animal is prepped with isopropyl alcohol followed by dilute Betadine solution and then draped in a sterile fashion. A 22-gauge IV running Ringer's lactate at 20 ml/h is placed in an ear vein. A standard 3-4 cm left anterolateral thoracotomy allows excellent exposure of the heart through the 4th intercostal space. For animals in the infarct group, the circumflex artery is ligated with a 6-0 Prolene suture. A carefully measured volume of injectate is given into the infarcted myocardium using a 30-gauge needle. The pneumothorax is evacuated prior to suture-closing the layers of the chest with absorbable suture. The rabbit is extubated once it is breathing appropriately. It is kept warm and given buprenorphine (0.05 mg/kg) for pain control.

Complexes containing pEpo-SV-VEGF-EpoUTR (SEQ ID NO:7) and a water soluble lipopolymer (WSLP) gene carrier are prepared at a 10/1 N/P ratio as described previously. M. Lee et al., Water soluble lipopolymer as an efficient carrier for gene delivery to myocardium, 10 Gene Ther. 585-593 (2003). The WSLP/plasmid complex is injected to the left ventricles of the hearts. All injections are performed over 1 min in a subepicardial location with a fixed amount of plasmid (50 μg) in injectate volumes of 500 μl. The hearts are harvested 4 days after the injections and homogenized in lysis buffer (Promega). Transgene expression is measured by ELISA using a ChemiKine human vascular endothelial growth factor sandwich ELISA kit (Chemicon, Temecula, Calif.). In all, 100 μl of the sample is added to the designated wells; 25 μl of biotinylated rabbit anti-human VEGF polyclonal antibody is added to each well, and the plate is incubated at room temperature for 3 h. After the incubation, the plate is washed five times with a wash buffer. Then, 50 μl of streptavidin-alkaline phosphatase is added to each well and the plate is incubated at room temperature for 45 min. The substrate is added to the wells and the absorbance is measured at 490 nm.

### EXAMPLE 7

Application of the Epo Enhancer/3′-UTR Combination System to Cancer Treatment

A patient suffering ischemic disease, such as cardiac ischemia or hindlimb ischemia, is treated with pEpo-SV-VEGF-EpoUTR, typically complexed with a pharmaceutically acceptable gene delivery carrier, by administering the plasmid by injection into the ischemic muscle, such as the myocardium or muscles of the hind limb.

### EXAMPLE 8

Application of the Epo Enhancer/3′-UTR Combination System to Cancer Treatment

Another application of this system is hypoxia-specific cancer therapy. Hypoxic regions are found in many solid tumors. Therefore, this highly specific hypoxia gene expression system is applicable to cancer gene therapy by cloning of DNA coding for anticancer agents into an expression plasmid, such as by replacing the VEGF coding sequence of pEpo-SV-VEGF-EpoUTR with the coding sequence for the anticancer agent. The plasmid, typically complexed with a pharmaceutically acceptable gene delivery carrier, is administered into or near the solid tumor.

The subject matter claimed is:

1. The plasmid pEpo-SV-VEGF-EpoUTR (SEQ ID NO:7).

2. A reporter plasmid for testing expression of a luciferase coding sequence under hypoxic conditions, wherein the reporter plasmid is a member selected from the group consisting of pSV-Luc-EpoUTR (SEQ ID NO:5) and pEpo-SV-Luc-EpoUTR (SEQ ID NO:6).

4. A plasmid comprising a hypoxia-regulated enhancer element operationally configured adjacent to a promoter operable in mammalian cells, an expression cassette encoding vascular endothelial growth factor, and a 3′ untranslated region from a hypoxia-regulated gene, wherein expression of vascular endothelial growth factor in a suitable cell is higher under hypoxia as compared to normal oxygen tension.

5. The plasmid of claim 4 wherein the hypoxia-regulated enhancer element comprises an erythropoietin enhancer.

6. The plasmid of claim 4 wherein the promoter comprises an SV40 promoter.

7. The plasmid of claim 4 wherein the 3′ untranslated region is from the erythropoietin gene.

8. A composition comprising a mixture of pEpo-SV-VEGF-EpoUTR and a pharmaceutically acceptable gene delivery carrier.

9. A method for treating an ischemic disease comprising administering to a patient in need of treatment for such ischemic disease a composition comprising a mixture of pEpo-SV-VEGF-EpoUTR and a pharmaceutically acceptable gene delivery carrier.

10. The method of claim 9 wherein the ischemic disease is ischemic heart disease, and the mixture is injected into the myocardium.

11. The method of claim 9 wherein the ischemic disease is hindlimb ischemia, and the mixture is injected into a muscle of the hind limb.

12. A method for treating cancer in a patient having a solid tumor, comprising administering a plasmid comprising a hypoxia-regulated enhancer element operationally configured adjacent to a promoter operable in mammalian cells, an expression cassette encoding an anticancer agent, and a 3′ untranslated region from a hypoxia-regulated gene, wherein expression of the anticancer agent in an ischemic region of the solid tumor is higher than in non-ischemic tissues.


SEQUENCE LISTING (7 sequences)

SEQ ID NO: 1 - DNA, 5010 bp, Artificial Sequence
  pGL3-Promoter Vector
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 2 - DNA, 50 bp, Homo sapiens
gccctacgtgctgtctcacacagcctgtctgacctctcgacctaccggcg

SEQ ID NO: 3 - DNA, 588 bp, Homo sapiens
aagcttatgaactttctgctgtcttgggtgcattggagccttgccttgctgctctacctccaccatgccaagtggtcccaggctgcacccatggcagaaggaggggggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgatctaga

SEQ ID NO: 4 - DNA, 5175 bp, Artificial sequence
  Plasmid pEPO-SV-Luc.
ggtaccgagctcttacgcgtgctagcccgggctcgagatccgccggtaggtcgagaggtcagacaggctgtgtgagacagcacgtagggctctagagccctacgtgctgtctcacacagcctgtctgacctctcgacctaccggcgggatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 5 - DNA, 5705 bp, Artificial sequence
  Plasmid pSV-Luc-EpoUTR.
ggtaccgagctcttacgcgtgctagcccgggctcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagaccaggtgtgtccacctgggcatatccaccacctccctcaccaacattgcttgtgccacaccctcccccgccactcctgaaccccgtcgaggggctctcagctcagcgccagcctgtcccatggacactccagtgccaccaatgacatctcaggggccagaggaactgtccagagagcaactctgagatctaaggatgtcacagggccaacttgagggcccagagcaggaagcattcagagagcagctttaaactcagggacagacccatgctgggaagacgcctgagctcactcggcaccctgcaaaattgatgccaggacacgctttggaggcgatttacctgttttcgcacctaccatcagggacaggatgacctggagaacttaggtggcaagctgtgacttctccaggtctcacgggcatgggcactcccttggtggcaagagcccccttgacaccggggtggtgggaaccatgaagacaggatgggggctggcctctggctctcatggggtccaacttttgtgtattcttcaacctcattgacaagaactgaaaccaccaatatgactcttggcttttctgttttctgggaacctccaaatcccctggctctgtcccactcctggcagcatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 6 - DNA, 5816 bp, Artificial sequence
  Plasmid pEPO-SV-Luc-EpoUTR.
ggtaccgagctcttacgcgtgctagcccgggctcgagatccgccggtaggtcgagaggtcagacaggctgtgtgagacagcacgtagggctctagagccctacgtgctgtctcacacagcctgtctgacctctcgacctaccggcgggatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagaccaggtgtgtccacctgggcatatccaccacctccctcaccaacattgcttgtgccacaccctcccccgccactcctgaaccccgtcgaggggctctcagctcagcgccagcctgtcccatggacactccagtgccaccaatgacatctcaggggccagaggaactgtccagagagcaactctgagatctaaggatgtcacagggccaacttgagggcccagagcaggaagcattcagagagcagctttaaactcagggacagacccatgctgggaagacgcctgagctcactcggcaccctgcaaaattgatgccaggacacgctttggaggcgatttacctgttttcgcacctaccatcagggacaggatgacctggagaacttaggtggcaagctgtgacttctccaggtctcacgggcatgggcactcccttggtggcaagagcccccttgacaccggggtggtgggaaccatgaagacaggatgggggctggcctctggctctcatggggtccaacttttgtgtattcttcaacctcattgacaagaactgaaaccaccaatatgactcttggcttttctgttttctgggaacctccaaatcccctggctctgtcccactcctggcagcatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 7 - DNA, 4709 bp, Artificial
  Plasmid pEPO-SV-VEGF-EpoUTR.
ggtaccgagctcttacgcgtgctagcccgggctcgagatccgccggtaggtcgagaggtcagacaggctgtgtgagacagcacgtagggctctagagccctacgtgctgtctcacacagcctgtctgacctctcgacctaccggcgggatctgcgatctaagtaagcttatgaactttctgctgtcttgggtgcattggagccttgccttgctgctctacctccaccatgccaagtggtcccaggctgcacccatggcagaaggaggggggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgatctagaccaggtgtgtccacctgggcatatccaccacctccctcaccaacattgcttgtgccacaccctcccccgccactcctgaaccccgtcgaggggctctcagctcagcgccagcctgtcccatggacactccagtgccaccaatgacatctcaggggccagaggaactgtccagagagcaactctgagatctaaggatgtcacagggccaacttgagggcccagagcaggaagcattcagagagcagctttaaactcagggacagacccatgctgggaagacgcctgagctcactcggcaccctgcaaaattgatgccaggacacgctttggaggcgatttacctgttttcgcacctaccatcagggacaggatgacctggagaacttaggtggcaagctgtgacttctccaggtctcacgggcatgggcactcccttggtggcaagagcccccttgacaccggggtggtgggaaccatgaagacaggatgggggctggcctctggctctcatggggtccaacttttgtgtattcttcaacctcattgacaagaactgaaaccaccaatatgactcttggcttttctgttttctgggaacctccaaatcccctggctctgtcccactcctggcagcatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata